(19) United States
(12) Patent Application Publication
(10) Pub. No.: US 2023/0416257 A1
(43) Pub. Date: Dec. 28, 2023

(54) TOLL-LIKE RECEPTOR AGONIST

(71) Applicant: University of Montana, Missoula, MT (US)

(72) Inventor: Helene Bazin-Lee, Missoula, MT (US)

(21) Appl. No.: 18/038,291

(22) PCT Filed: Dec. 9, 2021
(86) PCT No.: PCT/US2021/062641

§ 371 Date: May 23, 2023

Related U.S. Application Data
(60) Provisional application No. 63/123,239, filed on Dec. 9, 2020.

(51) Int. Cl. C07D 487/04; C07J 43/00; C07F 9/6558; A61K 39/39

(52) U.S. Cl. CPC C07D 487/04; C07J 43/003; C07F 9/65583; A61K 39/39; A61K 2039/55511

(57) ABSTRACT

The present disclosure provides compounds of formula (I), (Ia), and (Ib), squaryl group- containing toll-like receptor ligands Also provided are pharmaceutical compositions and vaccine composition thereof and methods of treating a disease of disorder with the compounds and composition described herein.

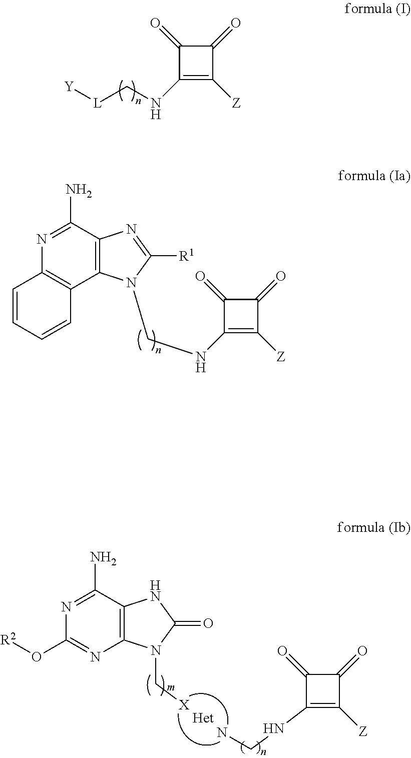

### CROSS-REFERENCE TO RELATED APPLICATIONS)

This application claims the benefit of U.S. Provisional Application No. 63/123,239 filed Dec. 9, 2020, the content of which is herein incorporated by reference in its entirety.

### FIELD

The present disclosure provides toll-like receptor ligands containing a squaryl group and methods of use thereof, for example as vaccine adjuvants.

### BACKGROUND

Worldwide, cancer is the second leading cause of death. In 2018, an estimated 9.6 million people died of cancer. Despite the promise of immunotherapy, a recent analysis estimated that only approximately 12.5% of all cancer patients in the US may respond to checkpoint inhibitors. One solution to treating cancers that are refractory to immunotherapy is to generate synergistic combination immunotherapeutics. Toll-Like Receptor (TLR) agonists, potent immune stimulators, have shown considerable promise when used in combination with checkpoint inhibitors in pre-clinical cancer models.

The development of novel vaccine adjuvants and immunotherapies based on TLR ligands has been a rapidly expanding area of research with clinical success across multiple indications and several approved products. One of the most promising classes of immunotherapeutics includes compounds targeting TLR7/8. Early versions of these TLR7/8 ligands rapidly enter the blood stream leading to systemic toxicity thereby limiting their use to topical administration. A second generation of lipidated TLR7/8 ligands greatly reduced the systemic toxicity of the core compounds leading to renewed commercial interest and ongoing clinical trials across multiple fields of use.

### SUMMARY

In one aspect, disclosed herein are compounds of formula (I):

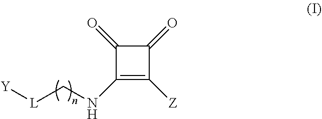

or a pharmaceutically acceptable salt thereof, wherein,

n is an integer from 2 to 6;

Y is

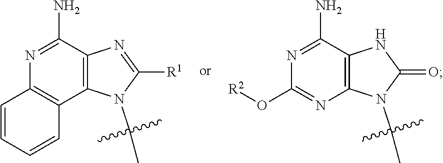

R¹ is hydrogen, C₁-C₆ alkyl, C₁-C₆ alkylamino, or C₁-C₆ alkoxy;

R² is C₁-C₈ alkyl;

L is a bond or

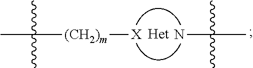

m is an integer from 1 to 6;

X is CH or N;

Het is a four-, five-, or six-membered saturated heterocycle;

Z is

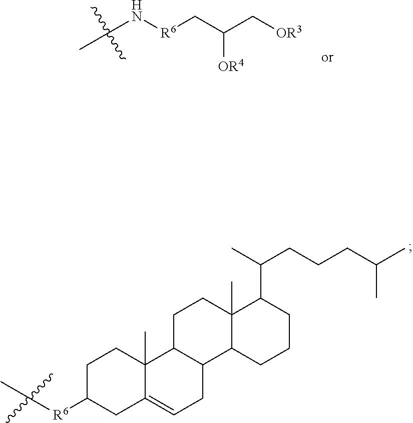

R³ is C₆-C₂₀ alkyl, C₆-C₂₀ alkenyl, or C(O)R⁵;

R⁴ is hydrogen, C₆-C₂₀ alkyl, C₆-C₂₀ alkenyl, or C(O)R⁵;

R⁵ C₆-C₂₀ alkyl or C₆-C₂₀ alkenyl;

R⁶ is (C₂-C₆ alkylene-O)ₚ or a bond; and

p is 0, 1, 2, 3, 4, 5, 6, 7, 8, 9, 10 ,11, 12, 13, 14, 15, 16, 17, 18, 19 or 20,

wherein

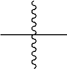

is the point of attachment to formula (I).

Also disclosed herein are compounds of formula (Ia), (Ib), and pharmaceutically acceptable salts thereof, along with pharmaceutical compositions and vaccine compositions comprising the compounds, methods for inducing an enhanced immune response in a subject using the compounds and compositions and methods of treating a disease or disorder using the compounds and compositions.

Other aspects and embodiments of the disclosure will be apparent in light of the following detailed description and accompanying figures.

### BRIEF DESCRIPTION OF THE DRAWINGS

FIG. 1 is graphs of TLR7 and TLR8 activity of an exemplary compound disclosed herein in comparison to other known TLR 7/8 ligands.

FIG. 2 is graphs of the induction of TNFα and IFNα in peripheral blood mononuclear cells (PBMCs) as a result of treatment with liposomal forms of an exemplary compound disclosed herein and other known TLR 7/8 ligands.

FIG. 3 is graphs of antibody induction in mice immunized with an exemplary compound disclosed herein.

FIG. 4 is graphs of cytokine secretion by antigen-stimulated T cells from draining lymph nodes (LNs) and spleen in mice immunized with an exemplary compound disclosed herein.

### DETAILED DESCRIPTION

Described herein are toll-like receptor ligands containing a squaryl group. These compounds overcome synthetic and stability challenges of known toll-like receptor ligands which result in unreproducible low yields, production of several unwanted by-products, and instability in acidic conditions.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, immunology, vaccination and cancer described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular. All publications, patent applications, patents and other references mentioned herein are incorporated by reference in their entirety.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} ED., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆ alkyl” or “C₁-C₆ alkyl” means a straight or branched chain hydrocarbon containing from 1 to 6 carbon atoms. The term “C₁₋₄ alkyl” or “C₁-C₄ alkyl” means a straight or branched chain saturated hydrocarbon containing from 1 to 4 carbon atoms.

The term “alkylamino,” as used herein, means at least one alkyl group, as defined herein, is appended to the parent molecular moiety through an amino group, as defined herein.

The term “alkenyl,” as used herein, means a straight or branched, hydrocarbon chain containing at least one carbon-carbon double bond and from 1 to 10 carbon atoms. Examples of alkenyl groups include, but are not limited to, ethenyl, propenyl, n-butenyl, i-butenyl, 3-methylbut-2-enyl, n-pentenyl, heptenyl, octenyl, decenyl, and the like.

The terms “heterocycle,” “heterocyclic” or “heterocyclyl” refer generally to ring systems containing at least one heteroatom as a ring atom where the heteroatom is selected from oxygen, nitrogen, and sulfur. Heterocycles may be a monocyclic heterocycle, a fused bicyclic heterocycle, a fused tricyclic heterocycle or a spiro heterocycle. The monocyclic heterocycle is generally a 4, 5, 6, 7, or 8-membered non-aromatic ring containing at least one heteroatom selected from O, N, or S. The 4-membered ring contains one heteroatom and optionally one double bond. The 5-membered ring contains zero or one double bond and one, two or three heteroatoms. The 6, 7, or 8-membered ring contains zero, one, or two double bonds, and one, two, or three heteroatoms. Representative examples of monocyclic heterocycle include, but are not limited to, azetidinyl, azepanyl, diazepanyl, 1,3-dioxanyl, 1,4-dioxanyl, 1,3- dioxolanyl , 4,5-dihydroisoxazol-5-yl, 3,4-dihydropyranyl, 1,3-dithiolanyl, 1,3-dithianyl, homomorpholinyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydrothienyl, thiadiazolinyl, thiadiazolidinyl, thiazolinyl, thiazolidinyl, thiomorpholinyl, 1,1-dioxidothiomorpholinyl, thiopyranyl, and trithianyl. The fused bicyclic heterocycle is a 7-12-membered ring system having a monocyclic heterocycle fused to a phenyl, to a saturated or partially saturated carbocyclic ring, or to another monocyclic heterocyclic ring, or to a monocyclic heteroaryl ring. Representative examples of fused bicyclic heterocycle include, but are not limited to, pyrrolopyridinyl, pyrazolopyridinyl, 4,5,6,7-tetrahydro-1H-indolyl, 4,5,6,7-tetrahydro-1H-indazolyl, 1,3-benzodioxol-4-yl, 1,3-benzodithiolyl, 3-azabicyclo[3. 1.0]hexanyl, hexahydro-1H-furo[3,4-c]pyrrolyl, 2,3-dihydro-1,4-benzodioxinyl, 2,3-dihydro-1H-indolyl, 5,6,7,8-tetrahydroimidazo[1,2-a]pyrazinyl, and 1,2,3,4-tetrahydroquinolinyl. The fused tricycle heterocycle is an 11-14-membered ring system having a monocyclic heterocycle fused to at least two of: a phenyl, a saturated or partially saturated carbocyclic ring, another monocyclic heterocyclic ring, or a monocyclic heteroaryl ring. Representative examples of fused tricyclic heterocycle include, but are not limited to, 3,4,5,6,7,8-hexahydro-pyrido-indolyl, 1, 2, 3, 4, 6,7,8- octahydro-carbazole and 1,2,3,4-tetrahydro-carbazole. Spiro heterocycle means a 4-, 5-, 6-, 7-, or 8-membered monocyclic heterocycle ring wherein two of the substituents on the same carbon atom form a second ring having 3, 4, 5, 6, 7, or 8 members. Examples of a spiro heterocycle include, but are not limited to, 1,4-dioxa-8-azaspiro[4.5]decanyl, 2-oxa-7-azaspiro[3.5]nonanyl, 2-oxa-6-azaspiro[3.3]heptanyl, and 8-azaspiro[4.5]decane. The monocyclic heterocycle groups of the present invention may contain an alkylene bridge of 1, 2, or 3 carbon atoms, linking two nonadjacent atoms of the group. Examples of such a bridged heterocycle include, but are not limited to, 2,5-diazabicyclo[2.2.1]heptanyl, 2-azabicyclo[2.2.1]heptanyl, 2-azabicyclo[2.2.2]octanyl, and oxabicyclo[2.2.1]heptanyl. The monocyclic, fused bicyclic, and spiro heterocycle groups are connected to the parent molecular moiety through any substitutable carbon atom or any substitutable nitrogen atom contained within the group.

Terms such as “alkyl,” “cycloalkyl,” “alkylene,” “cycloalkylene,” etc. may be preceded by a designation indicating the number of atoms present in the group in a particular instance (e.g.,“C₁-C₄ alkyl,” “C₁-C₄ alkylene”). These designations are used as generally understood by those skilled in the art. For example, the representation “C” followed by a subscripted number indicates the number of carbon atoms present in the group that follows. Thus, “C₃ alkyl” is an alkyl group with three carbon atoms (e.g., n-propyl, isopropyl). Where a range is given, as in “C₁₋₄,” the members of the group that follows may have any number of carbon atoms falling within the recited range. A “C₁-C₄ alkyl,” for example, is an alkyl group having from 1 to 4 carbon atoms, however arranged (e.g., straight chain or branched).

If substituents are described as being independently selected from a group, each substituent is selected independent of the other. Each substituent, therefore, may be identical to or different from the other substituent(s).

The term “−” designates a single bond (−) or a double bond (═).

The term

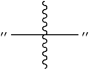

designates the point of attachment to the parent molecule.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Thus, included within the scope of the invention are tautomers of the compounds disclosed herein. The structures also include zwitterionic forms of the compounds or salts of the compounds disclosed herein, where appropriate.

As used herein, the term “preventing” refers to partially or completely delaying onset of an infection, disease, disorder and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, “treat,” “treating,” and the like means a slowing, stopping, or reversing of progression of a disease or disorder when provided a composition described herein to an appropriate control subject. The term also means a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, the terms “providing,” “administering,” and “introducing” are used interchangeably herein and refer to the placement of the compositions of the disclosure into a subject by a method or route which results in at least partial localization of the composition to a desired site. The compositions can be administered by any appropriate route which results in delivery to a desired location in the subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. COMPOUNDS

In some aspects, disclosed is a compound of formula (I):

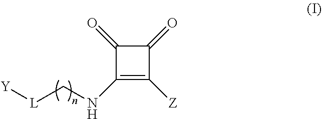

or a pharmaceutically acceptable salt thereof, wherein,

n is an integer from 2 to 6;

Y is

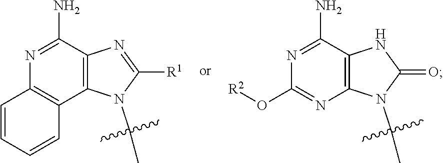

R¹ is hydrogen, C₁-C₆ alkyl, C₁-C₆ alkylamino, or C₁-C₆ alkoxy;

R² is C₁-C₈ alkyl;

L is a bond or

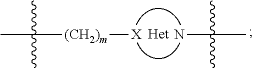

m is an integer from 1 to 6;

X is CH or N;

Het is a four-, five-, or six-membered saturated heterocycle;

Z is

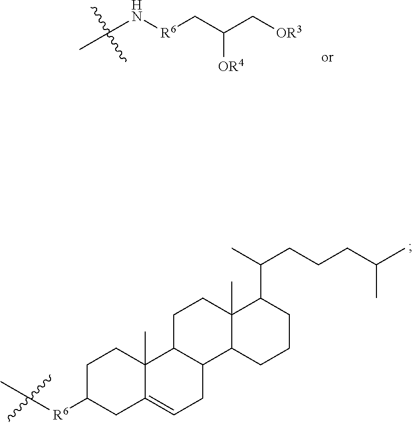

R³ is C₆-C₂₀ alkyl, C₆-C₂₀ alkenyl, or C(O)R⁵;

R⁴ is hydrogen, C₆-C₂₀ alkyl, C₆-C₂₀ alkenyl, or C(O)R⁵;

R⁵ C₆-C₂₀ alkyl or C₆-C₂₀ alkenyl;

R⁶ is (C₂-C₆ alkylene-O)ₚ or a bond; and

p is 0, 1, 2, 3, 4, 5, 6, 7, 8, 9, 10 ,11, 12, 13, 14, 15, 16, 17, 18, 19 or 20,

wherein

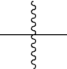

is the point of attachment to formula (I).

In some embodiments, R⁶ is (C₂-C₆ alkylene-O)ₚ.

In some embodiments, Z is

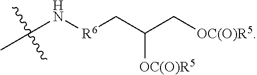

In select embodiments, Z is

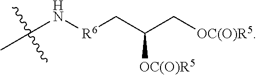

In some embodiments, R⁵ is C₆-C₂₀ alkyl. In select embodiments, R⁵ is C₁₀-C₂₀ alkyl.

In select embodiments, Z is

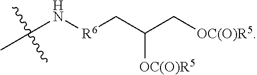

and R⁶ is (C₂-C₆ alkylene-O)ₚ.

In some embodiments, R⁵ is C₆-C₂₀ alkyl. In select embodiments, R⁵ is C₁₀-C₂₀ alkyl.

In select embodiments, Z is

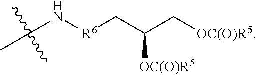

In some embodiments, Z is

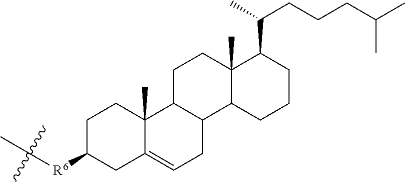

In select embodiments, Z is

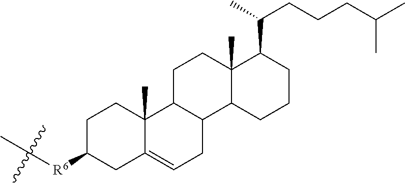

and R⁶ is (C₂-C₆ alkylene-O)ₚ.

In some embodiments, p is 0, 1, 2, 3, 4, 5, or 6.

In some embodiments, Y is

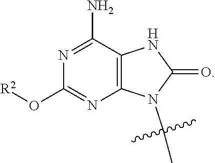

In some embodiments, R² is C₄-C₈ alkyl.

In some embodiments, L is

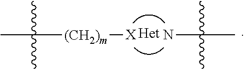

In some embodiments, m is 1, 2, 3, 4, or 5. In some embodiments, X is CH or N. In some embodiments, Het is a five- or six-membered saturated heterocycle.

In some embodiments, L is

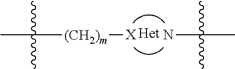

and m is 1, 2, 3, 4, or 5. In some embodiments, L is

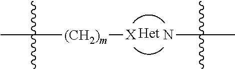

and X is CH or N. In some embodiments, L is

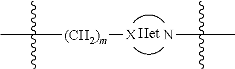

and Het is a five- or six-membered saturated heterocycle.

In some embodiments, L is

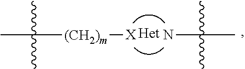

m is 1, 2, 3, 4, or 5, and X is CH or N. In some embodiments, L is

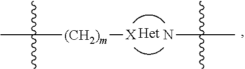

m is 1, 2, 3, 4, or 5, and Het is a five-or six-membered saturated heterocycle. In some embodiments, L is

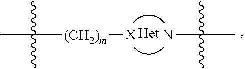

m is 1, 2, 3, 4, or 5, Xis CH or N, and Het is a five- or six-membered saturated heterocycle.

In select embodiments, L is

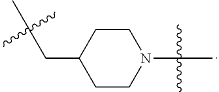 [SMILES: *CC1CCN(*)CC1]

In some embodiments, Y is

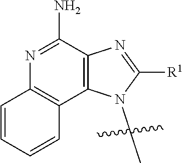

and R¹ is C₁-C₆ alkyl.

In another aspect, disclosed is a compound of formula (Ia):

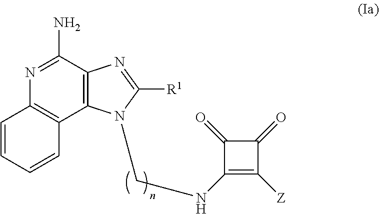

or a pharmaceutically acceptable salt thereof, wherein,

n is an integer from 2 to 6;

R¹ is hydrogen, C₁-C₆ alkyl, C₁-C₆ alkylamino, or C₁-C₆ alkoxy;

Z is

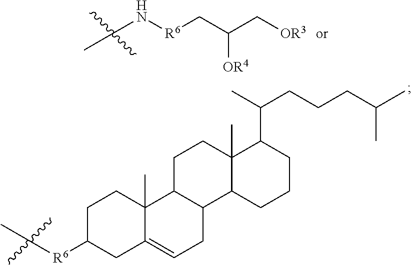

R³ is C₆-C₂₀ alkyl, C₆-C₂₀ alkenyl, or COR⁵;

R⁴ is hydrogen, C₆-C₂₀ alkyl, C₆-C₂₀ alkenyl, or COR⁵;

R⁵ is C₆-C₂₀ alkyl or C₆-C₂₀ alkenyl;

R⁶ is (C₂-C₆ alkylene-O)ₚ or a bond; and

p is 0, 1, 2, 3, 4, 5, 6, 7, 8, 9, 10 ,11, 12, 13, 14, 15, 16, 17, 18, 19 or 20,

wherein

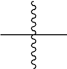

is the point of attachment to formula (Ia).

In some embodiments, R¹ is C₁-C₆ alkyl.

In some embodiments, R⁶ is (C₂-C₆ alkylene-O)ₚ.

In some embodiments, Z is

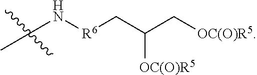

In some embodiments, R⁵ is

C₆-C₂₀ alkyl. In select embodiments, R⁵ is C₁₀-C₂₀ alkyl.

In select embodiments, Z is

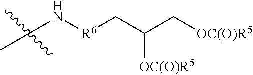

and R⁶ is (C₂-C₆ alkylene-O)ₚ.

In some embodiments, R⁵ is C₆-C₂₀ alkyl. In select embodiments, R⁵ is C₁₀-C₂₀ alkyl.

In select embodiments, Z is

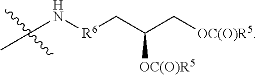

In some embodiments, Z is

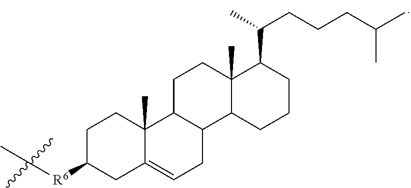

In select embodiments, Z is

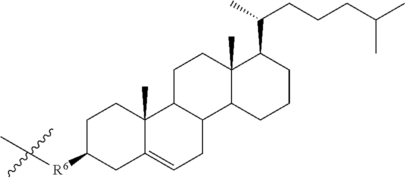

and R⁶ is (C₂-C₆ alkylene-O)ₚ.

In some embodiments, p is 0, 1, 2, 3, 4, 5, or 6.

In another aspect, disclosed is a compound of formula (Ib):

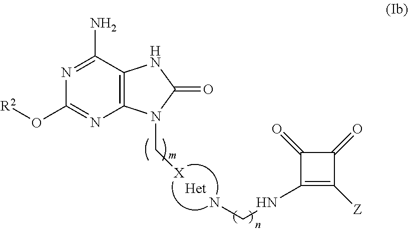

or a pharmaceutically acceptable salt thereof, wherein,

n is an integer from 2 to 6;

R² is C₁-C₈ alkyl;

m is an integer from 1 to 6;

X is CH or N;

Het is a four-, five-, or six-membered saturated heterocycle;

Z is

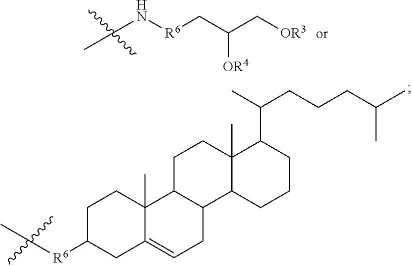

R³ is C₆-C₂₀ alkyl, C₆-C₂₀ alkenyl, or COR⁵;

R⁴ is hydrogen, C₆-C₂₀ alkyl, C₆-C₂₀ alkenyl, or COR⁵;

R⁵ is C₆-C₂₀ alkyl or C₆-C₂₀ alkenyl;

R⁶ is (C₂-C₆ alkylene-O)ₚ or a bond; and

p is 0, 1, 2, 3, 4, 5, 6, 7, 8, 9, 10 ,11, 12, 13, 14, 15, 16, 17, 18, 19 or 20,

wherein

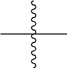

is the point of attachment to formula (Ib).

In some embodiments, L is

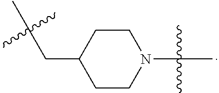 [SMILES: *CC1CCN(*)CC1]

In some embodiments, R⁶ is (C₂-C₆ alkylene-O)ₚ.

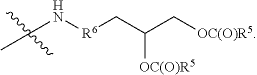

In some embodiments, Z is

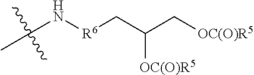

In some embodiments, R⁵ is C₆-C₂₀ alkyl. In select embodiments, R⁵ is C₁₀-C₂₀ alkyl.

In select embodiments, Z is and R⁶ is (C₂-C₆ alkylene-O)ₚ.

In some embodiments, R⁵ is C₆-C₂₀ alkyl. In select embodiments, R⁵ is C₁₀-C₂₀ alkyl.

In select embodiments, Z is

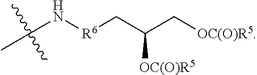

In some embodiments, Z is

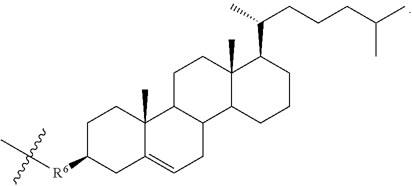

In select embodiments, Z is

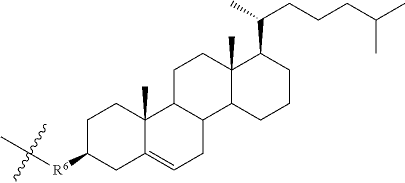

and R⁶ is (C₂-C₆ alkylene-O)ₚ.

In some embodiments, p is 0, 1, 2, 3, 4, 5, or 6.

In another aspect, disclosed is a compound selected from the group consisting of:

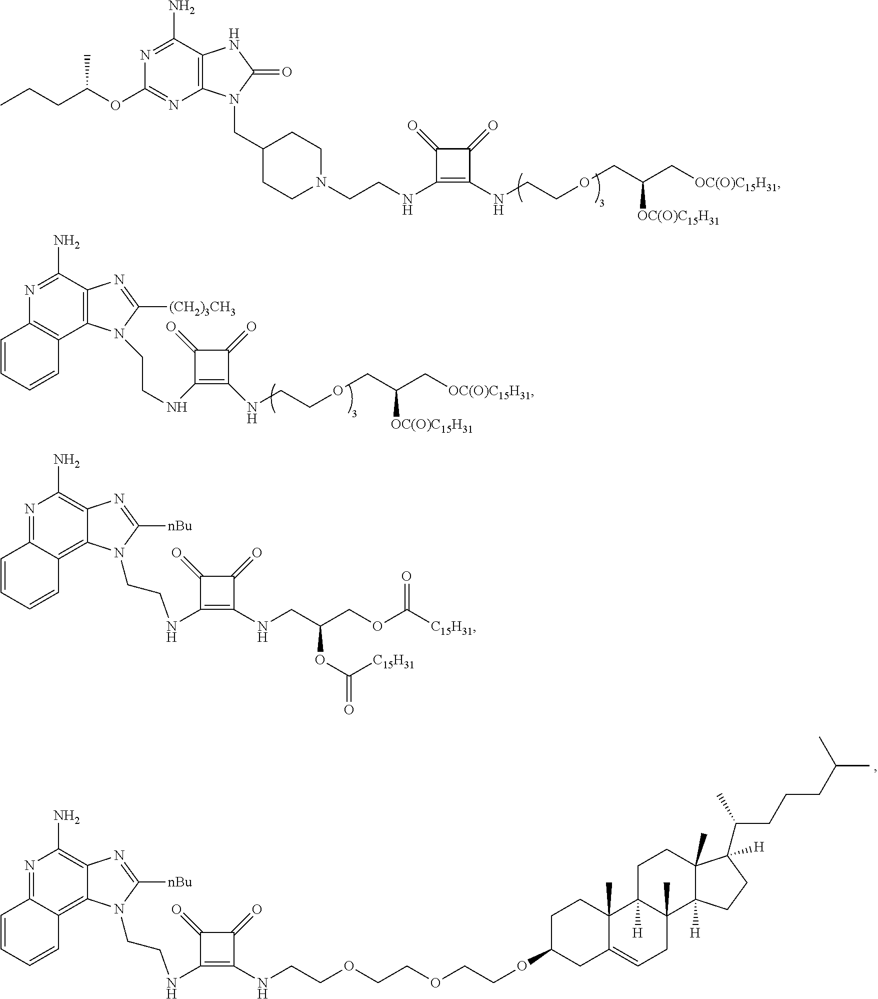

or a pharmaceutically acceptable salt thereof.

The compounds of the present disclosure may exist as stereoisomers wherein asymmetric or chiral centers are present. A stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers may include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Fumiss, Hannaford, Smith, and Tatchell, “Vogel's Textbook of Practical Organic Chemistry,” 5th edition (1989), Longman Scientific & Technical, Essex CM20 2JE, England, or (2) direct separation of the mixture of optical enantiomers on chiral chromatographic columns, or (3) fractional recrystallization methods.

It should be understood that the compounds of the present disclosure may possess tautomeric forms, as well as geometric isomers, and that these also constitute embodiments of the disclosure.

The present disclosure also may include isotopically-labeled compounds, which may be identical to those recited in formula (I), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸0, ¹⁷0, ³¹P, ³²P, ³⁵S, ¹⁸F, and ³⁶Cl, respectively. Substitution with heavier isotopes such as deuterium, e.g., ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ^{U}C, ¹³N, ¹⁵0, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

### a) Pharmaceutically Acceptable Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, l-ephenamine and N,N′-dibenzylethylenediamine, ethylenediamine, ethanolamine, diethanolamine, piperidine, piperazine, and the like.

### b) General Synthesis

Abbreviations which have been used in the descriptions of the Schemes that follow are: DIPEA, diisopropylethylamine.

The compositions described herein may be made by sequential coupling of a requisite oxoadenine or imidazoquinoline with commercially available squaryl diesters and a PEGylated amine, as shown for an exemplary oxoadenine in Scheme 1, where X¹ and X² are independently C₁-C₄ alkyl groups and all other constituents are as defined elsewhere herein. The PEGylated amine may be prepared from commercially available triethyleneglycol monochloride.

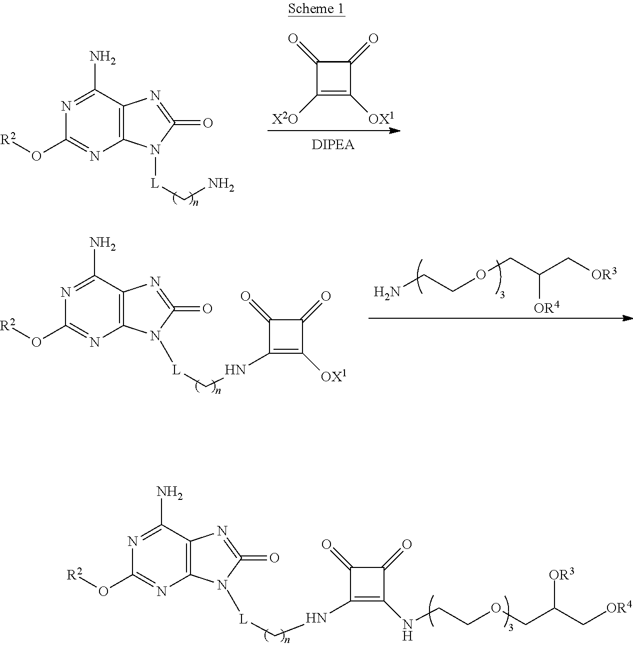

The compositions described herein may alternatively be made by sequential coupling of the commercially available squaryl diesters with a PEGylated amine and followed by the coupling of that intermediate to a requisite oxoadenine or imidazoquinoline, as shown in Scheme 2 for an exemplary imidazoquinoline, where X¹ and X² are independently C₁-C₄ alkyl groups and all other constituents are as defined elsewhere herein.

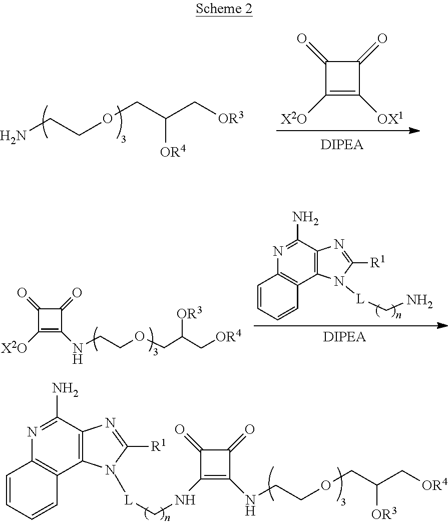

The compounds and intermediates may be isolated and purified by methods well-known to those skilled in the art of organic synthesis. Examples of conventional methods for isolating and purifying compounds may include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for each individual step may vary depending on the particular reactants employed and substituents present in the reactants used. Specific procedures are provided in the Examples section. Reactions may be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or may be prepared by one skilled in the art from commercially available materials using methods described in the chemical literature. Starting materials, if not commercially available, may be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4th ed.), John Wiley & Sons, NY (2006), which is incorporated herein by reference in its entirety. Synthesis of the compounds of the invention may be accomplished by methods analogous to those described in the synthetic schemes described hereinabove and in specific examples.

When an optically active form of a disclosed compound is required, it may be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it may be obtained by carrying out one of the above procedures using a pure geometric isomer as a starting material, or by resolution of a mixture of the geometric isomers of the compound or intermediates using a standard procedure such as chromatographic separation.

It will be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### 3. COMPOSITIONS

### a) Pharmaceutical Compositions

The compounds may also be incorporated into pharmaceutical compositions suitable for administration to a subject (such as a patient, which may be a human or non-human).

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. A carrier may include a single ingredient or a combination of two or more ingredients.

Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis). Techniques and formulations may generally be found in “Remington's Pharmaceutical Sciences,” (Meade Publishing Co., Easton, Pa.). Therapeutic compositions must typically be sterile and stable under the conditions of manufacture and storage.

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol.

Suitable lubricants include, but are not limited to, silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma.

Suitable binders include, but are not limited to, polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose.

Suitable disintegrants include, but are not limited to, agar, alginic acid and the sodium salt thereof, effervescent mixtures, croscarmelose, crospovidone, sodium carboxymethyl starch, sodium starch glycolate, clays, and ion exchange resins.

Suitable colorants include, but are not limited to, a colorant such as an FD&C dye. When used, the amount of colorant in a systemic or topical composition is typically about 0.005 to about 1.0%.

Suitable flavors include, but are not limited to, menthol, peppermint, and fruit flavors. The amount of flavor(s), when used, in a systemic or topical composition is typically about 0.1 to about 1.0%.

Suitable sweeteners include, but are not limited to, aspartame and saccharin. The amount of sweetener(s) in a systemic or topical composition is typically about 0.001 to about 1%.

Suitable antioxidants include, but are not limited to, butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include, but are not limited to, benzalkonium chloride, methyl paraben and sodium benzoate. The amount of preservative(s) in a systemic or topical composition is typically about 0.01 to about 5%.

Suitable glidants include, but are not limited to, silicon dioxide. The amount of glidant(s) in a systemic or topical composition is typically about 1 to about 5%.

Suitable solvents include, but are not limited to, water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include, but are not limited to, AVICEL RC-591 (from FMC Corporation of Philadelphia, PA) and sodium alginate. The amount of suspending agent(s) in a systemic or topical composition is typically about 1 to about 8%.

Suitable surfactants include, but are not limited to, lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp.587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

### b) Vaccine Compositions

The compounds may also be incorporated into vaccine compositions. The vaccine compositions may further include an antigen. Suitable antigens include microbial pathogens, bacteria, viruses, proteins, glycoproteins lipoproteins, peptides, glycopeptides, lipopeptides, toxoids, carbohydrates, and tumor-specific antigens. Mixtures of two or more antigens may be employed.

The vaccine compositions may include an “effective amount” of the disclosed compounds. In the context of a vaccine composition, an “effective amount” refers to an amount effective, at dosages and for periods of time necessary, to achieve the desired result (e.g., to potentiate an immune response to one or more antigens). The immune response can be measured, for example, by measuring antibody titers against an antigen, assessing the ability of a vaccine containing the compound to immunize a host in response to a disease or antigen challenge, etc. For example, administering an “effective amount” of a compound or composition to a subject increases one or more antibody titers by 10% or more over a nonimmune control, by 20% or more over a nonimmune control, by 30% or more over a nonimmune control, by 40% or more over a nonimmune control, by 50% or more over a nonimmune control, by 50% or more over a nonimmune control, by 70% or more over a nonimmune control, by 80% or more over a nonimmune control, by 90% or more over a nonimmune control, or by 100% or more over a nonimmune control.

Vaccine preparation is a well-developed art and general guidance in the preparation and formulation of vaccines is readily available from any of a variety of sources. One such example is New Trends and Developments in Vaccines, edited by Volier et al. University Park Press, Baltimore, Md., U.S.A. 1978.

The vaccine compositions of the present disclosure may also contain other compounds, which may be biologically active or inactive. For example, one or more immunogenic portions of other tumor antigens may be present, either incorporated into a fusion polypeptide or as a separate compound, within the vaccine composition. Vaccine compositions may generally be used for prophylactic and therapeutic purposes.

In one embodiment, the antigen in a vaccine composition is a peptide, polypeptide, or immunogenic portion thereof An “immunogenic portion,” as used herein is a portion of a protein that is recognized (e.g., specifically bound) by a B cell and/or T cell surface antigen receptor. Such immunogenic portions generally comprise at least 5 amino acid residues, more preferably at least 10, and still more preferably at least 20 amino acid residues of an antigenic protein or a variant thereof

Immunogenic portions of antigen polypeptides may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T cell lines or clones. As used herein, antisera and antibodies are “antigen-specific” if they specifically bind to an antigen (e.g., they react with the protein in an ELISA or other immunoassay, and do not react detectably with unrelated proteins). Such antisera and antibodies may be prepared as described herein, and using well known techniques. An immunogenic portion of a protein is a portion that reacts with such antisera and/or T cells at a level that is not substantially less than the reactivity of the full length polypeptide (e.g., in an ELISA and/or T cell reactivity assay). Such immunogenic portions may react within such assays at a level that is similar to or greater than the reactivity of the full length polypeptide. Such screens may generally be performed using methods well known to those of ordinary skill in the art, such as those described in Harlow and Lane, Antibodies: A Laboratory Manual, Cold Spring Harbor Laboratory, 1988. For example, a polypeptide may be immobilized on a solid support and contacted with patient sera to allow binding of antibodies within the sera to the immobilized polypeptide. Unbound sera may then be removed and bound antibodies detected using, for example, Protein A.

Peptide and polypeptide antigens may be prepared using any of a variety of well-known techniques. Recombinant polypeptides encoded by DNA sequences may be readily prepared from isolated DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast, and higher eukaryotic cells, such as mammalian cells and plant cells. Preferably, the host cells employed are E. coli, yeast or a mammalian cell line such as COS or CHO.

Portions and other variants of a protein antigen having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See, Merrifield, J. Am. Chem. Soc. 85:2149-2146, 1963. Equipment for automated synthesis of polypeptides is commercially available from suppliers such as Perkin Elmer/Applied BioSystems Division (Foster City, Calif), and may be operated according to the manufacturer's instructions.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al. , Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

In another embodiment, a compound or adjuvant composition described herein may be used in the preparation of DNA-based vaccine compositions. Illustrative vaccines of this type contain DNA encoding one or more polypeptide antigens, such that the antigen is generated in situ. The DNA may be present within any of a variety of delivery systems known to those of ordinary skill in the art, including nucleic acid expression systems, bacterial and viral expression systems. Numerous gene delivery techniques are well known in the art, such as those described by Rolland, Crit. Rev. Therap. Drug Carrier Systems 15: 143-198, 1998, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In one preferred embodiment, the DNA is introduced using a viral expression system (e.g., vaccinia or other pox virus, retrovirus, or adenovirus), which typically involves the use of a non-pathogenic (defective), replication competent virus. Illustrative systems are disclosed, for example, in Fisher-Hoch el al., Proc. Natl. Acad. Sci. USA 86:317-321, 1989; Flexner el al., Ann. NY. Acad. Sci. 569:86-103, 1989; Flexner et al., Vaccine 8: 17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Rolls et al., Proc. Natl. Acad. Sci. USA 91 :215-219, 1994; Kass-Eisler et al., Proc. Natl. Acad. Sci. USA 90: 11498-11502, 1993; Guzman et al., Circulation 88:2838-2848, 1993; and Guzman et al., Cir. Res. 73: 1202-1207, 1993. Techniques for incorporating DNA into such expression systems are well known to those of ordinary skill in the art.

A vaccine composition may contain pharmaceutically acceptable salts of the desired polynucleotide, polypeptide and/or carbohydrate antigens. For example, such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

The composition may exhibit strong adjuvant effects when administered over a wide range of dosages and a wide range of ratios.

The amount of antigen in each vaccine dose is generally selected as an amount which induces an immunoprotective response without significant adverse side effects in typical vaccines. Such amount will vary depending upon which specific immunogen is employed and how it is presented. Of course, the dosage administered may be dependent upon the age, weight, kind of concurrent treatment, if any, and nature of the antigen administered.

The immunogenic activity of a given amount of a vaccine composition can be readily determined, for example by monitoring the increase in titer of antibody against the antigen used in the vaccine composition (Dalsgaard, K. Acta Veterinia Scandinavica 69: 1-40 (1978)). Another common method involves injecting CD-1 mice intradermally with various amounts of a vaccine composition, later harvesting sera from the mice and testing for anti-immunogen antibody, e.g., by ELISA. These and other similar approaches will be apparent to the skilled artisan.

The antigen can be derived and/or isolated from essentially any desired source depending on the infectious disease, autoimmune disease, condition, cancer, pathogen, or a disease that is to be treated with a given vaccine composition. By way of illustration, the antigens can be derived from viral sources, such as influenza virus, feline leukemia virus, feline immunodeficiency virus, Human HIV-1, HIV-2, Herpes Simplex virus type 2, Human cytomegalovirus, Hepatitis A, B, C or E, Respiratory Syncytial virus, human papilloma virus rabies, measles, or hoof and mouth disease viruses. Illustrative antigens can also be derived from bacterial sources, such as anthrax, diphtheria, Lyme disease, malaria, tuberculosis, Leishmaniasis, T. cruzi, Ehrlichia, Candida, etc., or from protozoans such as Babeosis bovis or Plasmodium. The antigen(s) will typically be comprised of natural or synthetic amino acids, e.g., in the form of peptides, polypeptides, or proteins, can be comprised of polysaccharides, or can be mixtures thereof. Illustrative antigens can be isolated from natural sources, synthesized by means of solid phase synthesis, or can be obtained by way of recombinant DNA techniques.

In another embodiment, tumor antigens may be used in the vaccine compositions for the prophylaxis and/or therapy of cancer. Tumor antigens are surface molecules that are differentially expressed in tumor cells relative to non-tumor tissues. Tumor antigens make tumor cells immunologically distinct from normal cells and provide diagnostic and therapeutic targets for human cancers. Tumor antigens have been characterized either as membrane proteins or as altered carbohydrate molecules of glycoproteins or glycolipids on the cell surface. Cancer cells often have distinctive tumor antigens on their surfaces, such as truncated epidermal growth factor, folate binding protein, epithelial mucins, melanoferrin, carcinoembryonic antigen, prostate-specific membrane antigen, HER2-neu, which are candidates for use in therapeutic cancer vaccines. Because tumor antigens are normal or related to normal components of the body, the immune system often fails to mount an effective immune response against those antigens to destroy the tumor cells. To achieve such a response, the adjuvant systems described herein can be utilized. As a result, exogenous proteins can enter the pathway for processing endogenous antigens, leading to the production of cytolytic or cytotoxic T cells (CTL). This adjuvant effect facilitates the production of antigen specific CTLs which seek and destroy those tumor cells carrying on their surface the tumor antigen(s) used for immunization. Illustrative cancer types for which this approach can be used include prostate, colon, breast, ovarian, pancreatic, brain, head and neck, melanoma, leukemia, lymphoma, etc.

In one embodiment, the antigen present in the vaccine composition is not a foreign antigen, but a self-antigen, e.g., the vaccine composition is directed toward an autoimmune disease. Examples of autoimmune diseases include type 1 diabetes, conventional organ specific autoimmunity, neurological disease, rheumatic diseases/connective tissue disease, autoimmune cytopenias, and related autoimmune diseases. Such conventional organ specific autoimmunity may include thyroiditis (Graves+Hashimoto's), gastritis, adrenalitis (Addison's), ovaritis, primary biliary cirrhosis, myasthenia gravis, gonadal failure, hypoparathyroidism, alopecia, malabsorption syndrome, pernicious anemia, hepatitis, anti-receptor antibody diseases and vitiligo. Such neurological diseases may include schizophrenia, Alzheimer's disease, depression, hypopituitarism, diabetes insipidus, sicca syndrome and multiple sclerosis. Such rheumatic diseases/connective tissue diseases may include rheumatoid arthritis, systemic lupus erythematous (SLE) or Lupus, scleroderma, polymyositis, inflammatory bowel disease, dermatomyositis, ulcerative colitis, Crohn's disease, vasculitis, psoriatic arthritis, exfoliative psoriatic dermatitis, pemphigus vulgaris, Sjogren's syndrome. Other autoimmune related diseases may include autoimmune uvoretinitis, glomerulonephritis, post myocardial infarction cardiotomy syndrome, pulmonary hemosiderosis, amyloidosis, sarcoidosis, aphthous stomatitis, and other immune related diseases, as presented herein and known in the related arts.

The composition may include other polynucleotides and/or polypeptides. It will be apparent that a vaccine may contain pharmaceutically acceptable salts of the polynucleotides and polypeptides provided herein. Such salts may be prepared from pharmaceutically acceptable nontoxic bases, including organic bases (e.g., salts of primary, secondary and tertiary amines and basic amino acids) and inorganic bases (e.g., sodium, potassium, lithium, ammonium, calcium and magnesium salts).

The vaccine compositions may be formulated for any appropriate manner of administration, and thus administered, including for example, topical, oral, nasal, intravenous, intravaginal, epicutaneous, sublingual, intracranial, intradermal, intraperitoneal, subcutaneous, intramuscular administration, or via inhalation. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed.

The vaccine compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, vaccine compositions may be formulated as a lyophilizate. Compounds may also be encapsulated within liposomes using well known technology.

The vaccine compositions may also comprise other adjuvants or immunoeffectors. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); mineral salts (for example, aluminum, silica, kaolin, and carbon); aluminum salts such as aluminum hydroxide gel (alum), AlK(SO₄)₂, AlNa(SO₄)₂, AlNH (SO₄)₂, and Al(OH)₃; salts of calcium (e.g., Ca₃(PO₄)₂, ), iron or zinc; an insoluble suspension of acylated tyrosine; acylated sugars; cationically or anionically derivatized polysaccharides; polynucleotides (for example, poly IC and poly AU acids); polyphosphazenes; cyanoacrylates; polymerase-(DL-lactide-co-glycoside); biodegradable microspheres; liposomes; lipid A and its derivatives; monophosphoryl lipid A; wax D from Mycobacterium tuberculosis, as well as substances found in Corynebacterium parvum, Bordetella pertussis, and members of the genus Brucella); bovine serum albumin; diphtheria toxoid; tetanus toxoid; edestin; keyhole-limpet hemocyanin; Pseudomonal Toxin A; choleragenoid; cholera toxin; pertussis toxin; viral proteins; and Quil A. Aminoalkyl glucosamine phosphate compounds can also be used (see, e.g., WO 98/50399, U.S. Pat. No. 6,113,918 (which issued from U.S. Ser. No. 08/853,826), and U.S. Ser. No. 09/074,720). In addition, adjuvants such as cytokines (e.g., GM-CSF or interleukin-2, -7, or -12), interferons, or tumor necrosis factor, may also be used as adjuvants. Protein and polypeptide adjuvants may be obtained from natural or recombinant sources according to methods well known to those skilled in the art. When obtained from recombinant sources, the adjuvant may comprise a protein fragment comprising at least the immunostimulatory portion of the molecule. Other known immunostimulatory macromolecules which can be used include, but are not limited to, polysaccharides, tRNA, non-metabolizable synthetic polymers such as polyvinylamine, polymethacrylic acid, polyvinylpyrrolidone, mixed polycondensates (with relatively high molecular weight) of 4′,4-diaminodiphenylmethane-3,3′-dicarboxylic acid and 4-nitro-2- aminobenzoic acid (See, Sela, M., Science 166: 1365-1374 (1969)) or glycolipids, lipids or carbohydrates.

The compositions described herein may be administered as part of a sustained release formulation (e.g., a formulation such as a capsule, sponge, or gel (composed of polysaccharides, for example) that effects a slow release of compound following administration). Such formulations may generally be prepared using well known technology (see, e.g., Coombes et al., Vaccine 14: 1429-1438, 1996) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. Such carriers include microparticles of poly(lactide-co-glycolide), polyacrylate, latex, starch, cellulose, dextran and the like. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see, e.g., U.S. Pat. No. 5,151,254 and PCT applications WO 94/20078, WO/94/23701 and WO 96/06638). The amount of active compound contained within a sustained release formulation will vary depending upon the site of implantation, the rate and expected duration of release and the nature of the condition to be treated or prevented.

### 4. METHODS OF USE

### a) Modulating Immune Response

The disclosed compounds and compositions may be used in methods of modulating the immune response in a subject, the methods comprising administering to the subject an effective amount of a compound or composition described herein. In some embodiments, the subject may be suffering from a disease or disorder, e.g. cancer.

In some embodiments, disclosed compounds and compositions may be used in a method of inducing an enhanced immune response in a subject. The enhanced immune response may be induced by co-administering the compound or pharmaceutical composition with an antigen or by administration of the vaccine composition. Suitable antigens include microbial pathogens, bacteria, viruses, proteins, glycoproteins lipoproteins, peptides, glycopeptides, lipopeptides, toxoids, carbohydrates, and tumor-specific antigens, further described above. Mixtures of two or more antigens may be employed, such that the vaccine composition may be administered with another antigen.

In some embodiments, the disclosed compounds and compositions may be administered as a monotherapy. In other embodiments, the disclosed compositions may further contain at least one additional adjuvant or immunostimulant.

### b) Inducing or Enhancing Immunogenicity of an Antigen

The disclosed compounds and compositions may be used in methods of inducing or enhancing immunogenicity of an antigen in a subject, comprising administering to the subject an antigen and an effective amount of a compound or composition described herein.

Suitable antigens include microbial pathogens, bacteria, viruses, proteins, glycoproteins lipoproteins, peptides, glycopeptides, lipopeptides, toxoids, carbohydrates, and tumor-specific antigens. Mixtures of two or more antigens may be employed.

### c) Treatment of Disease or Disorder

The disclosed compounds and compositions may be used in methods of treating a disease or disorder.

In some embodiments, the disclosed compounds and compositions may be used in methods of treating cancer, or in methods of reducing or inhibiting the proliferation of cancer cells, the methods comprising administering to a subject in need thereof a therapeutically effective amount of a compound or a composition described herein.

Exemplary cancers may include the following: digestive/gastrointestinal cancers such as anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyosarcoma; islet cell pancreatic cancer; rectal cancer; and small intestine cancer; endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor; eye cancers such as intraocular melanoma; and retinoblastoma; musculoskeletal cancers such as Ewing's family of tumors; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma; breast cancer such as breast cancer including childhood and male breast cancer and breast cancer in pregnancy; neurologic cancers such as childhood brain stem glioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and childhood supratentorial primitive neuroectodermal tumors and pituitary tumor; genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor; Germ cell cancers such as childhood extracranial germ cell tumor; extragonadal germ cell tumor; ovarian germ cell tumor; head and neck cancers such as lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer; hematologic/blood cell cancers such as a leukemia (e.g., acute lymphoblastic leukemia including adult and childhood acute lymphoblastic leukemia; acute myeloid leukemia including adult and childhood acute myeloid leukemia; chronic lymphocytic leukemia; chronic myelogenous leukemia; and hairy cell leukemia); a lymphoma (e.g., AIDS-related lymphoma; cutaneous T cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sezary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders); lung cancer such as non-small cell lung cancer; and small cell lung cancer; respiratory cancers such as adult malignant mesothelioma; childhood malignant mesothelioma; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer; skin cancers such as Kaposi's sarcoma; Merkel cell carcinoma; melanoma; and childhood skin cancer; AIDS-related malignancies; other childhood cancers, unusual cancers of childhood and cancers of unknown primary site; and metastases of the aforementioned cancers.

The cancer may be a solid tumor. Examples of cancers that are solid tumors include, but are not limited to, brain, pancreatic, bladder, colon, non-small cell lung cancer (NSCLC), breast and ovarian cancers.

A wide range of second therapies may be used in conjunction with the compounds and compositions of the present disclosure. The second therapy may be a combination of a therapeutic agent or may be a second therapy not connected to administration of another agent. Such second therapies include, but are not limited to, surgery, immunotherapy, radiotherapy, or a second chemotherapeutic agent.

An “effective amount” refers to an amount effective, at dosages and for periods of time necessary, to achieve the desired therapeutic result. An effective amount of the composition may be determined by a person skilled in the art and may vary according to factors such as the disease state, age, sex, and weight of the individual, and the ability of the composition to elicit a desired response in the individual. An effective amount is also one in which any toxic or detrimental effects of a compound of the invention (e.g., a compound of formula (I)) are outweighed by the therapeutically beneficial effects.

The compounds or compositions disclosed herein may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies.

### 5. KITS

In one aspect, the disclosure provides kits comprising at least one disclosed compound or a pharmaceutically acceptable salt thereof, or a composition comprising the compound or a pharmaceutically acceptable salt thereof, and one or more of: at least one antigen; and instructions for administering the compound or composition.

In some embodiments, the at least one disclosed compound and the at least one antigen are co-formulated. In some embodiments, the at least one disclosed compound and the at least one antigen are co-packaged. The kits can also comprise compounds and/or products co- packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

The disclosed kits can be employed in connection with disclosed methods of use.

The kits may further include information, instructions, or both that use of the kit will provide increased immunity against certain pathogens in mammals (particularly humans). The information and instructions may be in the form of words, pictures, or both, and the like. In addition or in the alternative, the kit may include the compound, a composition, or both; and information, instructions, or both, regarding methods of administration of compound, or of the composition, preferably with the benefit of treating or preventing medical conditions in mammals (e.g., humans).

The compounds and processes of the disclosure may be better understood by reference to the following examples, which are intended as an illustration of and not a limitation upon the scope of the disclosure.

### 6. EXAMPLES

Abbreviations used in the examples that follow are:
Ac acetate
Boc tert-butoxycarbot protection group
Bu butyl
DIAD diisopropyl azodicarboxylate
DIPEA diisopropylethylamine
DMSO dimethyl sulfoxide
DPPA diphenylphosphoryl azide
DOPC 1,2-dioleoyl-sn-glycero-3-phosphocholine
Et ethyl
EtOAc ethyl acetate
EtOH ethanol
h hour
Pal palmitate or palmitic acid
Pd/C palladium on carbon
Pt/C platinum on carbon
PPh3 triphenylphosphine
Pyr pyridine
TEA triethylamine
TES triethylsilane
THF tetrahydrofuran
TsCl 4-Toluenesulfonyl chloride

### Example 1

### Compound Synthesis

Oxoadenine UM-2014 was prepared in 2 steps with good yield by sequential coupling of commercially available squaryl diester 3 with UM-3007 (See Evans et al., ACS Omega 2019 4 (13), 15665-15677, incorporated herein by reference in its entirety), and the requisite PEGylated amine analog 4. This synthesis was not water sensitive.

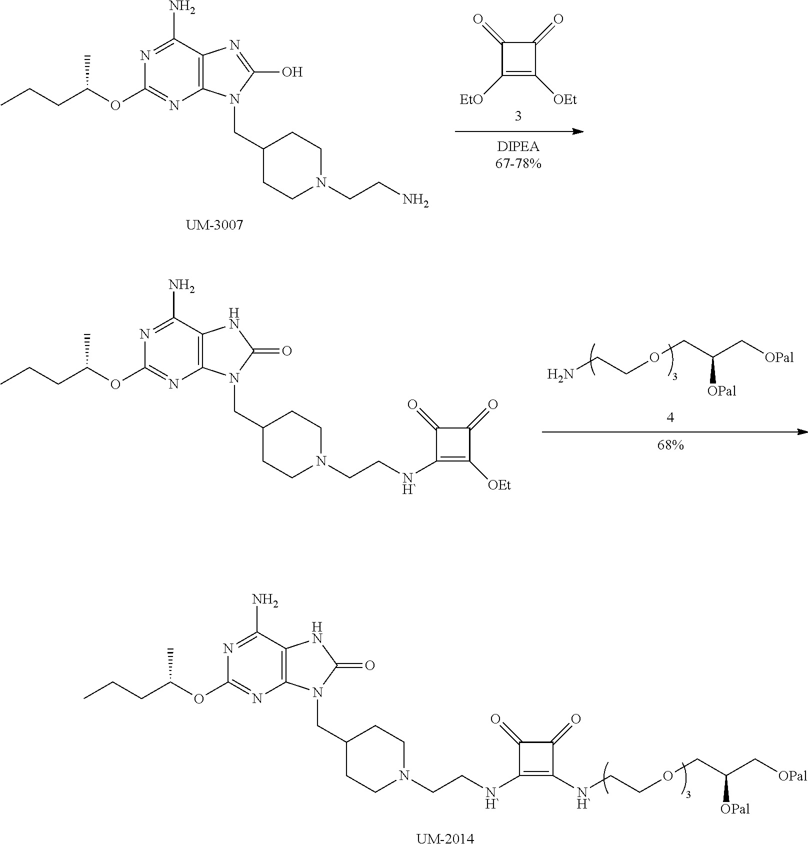

The PEGylated amine 4 is best prepared by azidation of dipalmitoyl PEGylated glycerol 5 (prepared in 5 steps from commercially available triethyleneglycol monochloride), followed by reduction of azide 6.

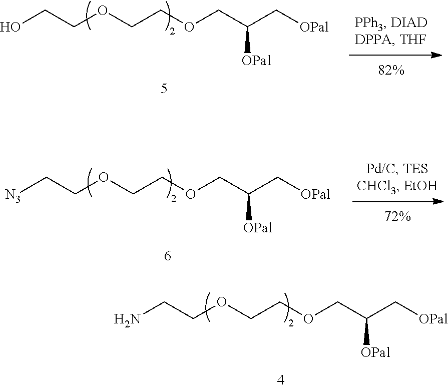

Imidazoquinoline UM-2018 was also prepared in good yield.

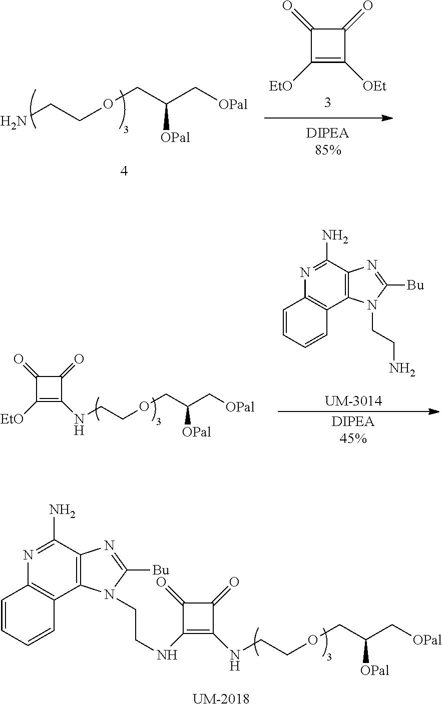

Imidazoquinoline UM-2027 was also prepared in good yield.

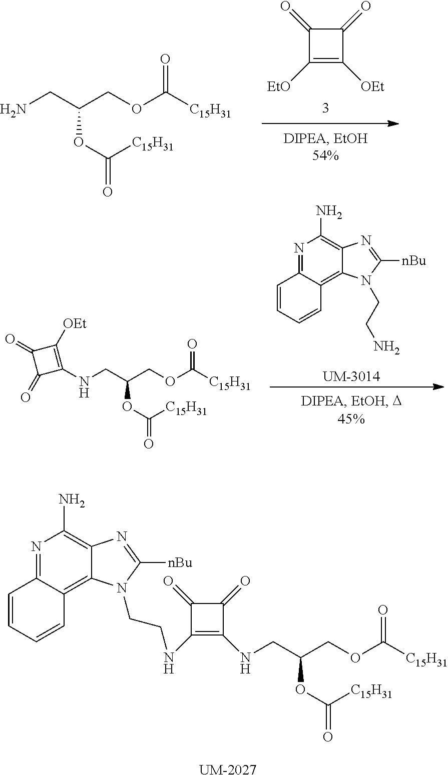

Imidazoquinoline UM-2028 was also prepared in good yield.

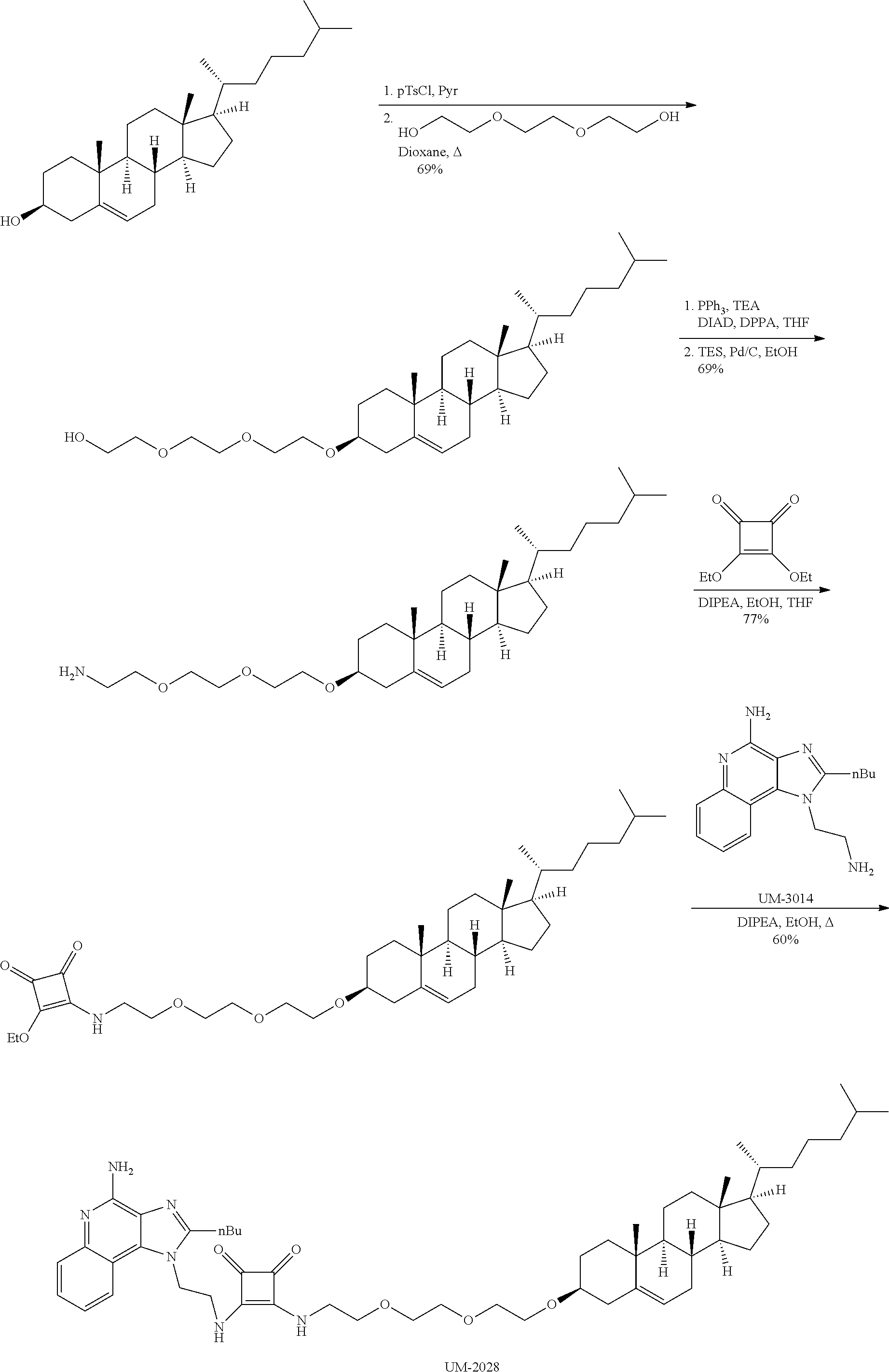

UM-3014 is preferably synthesized from 4-chloro-3-nitroquinoline, as shown below.

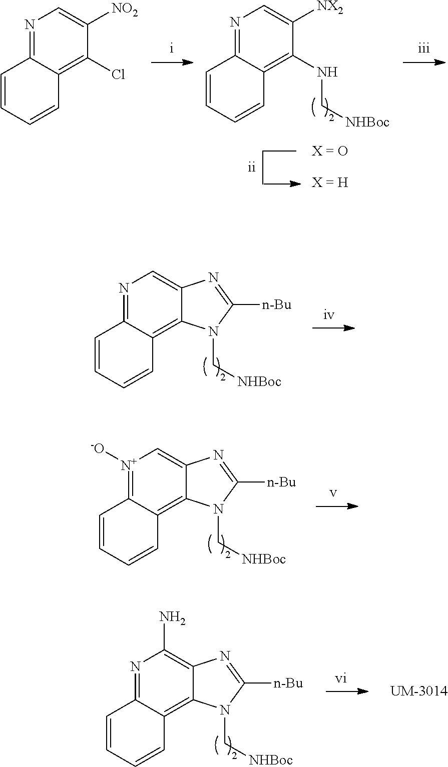

Reagents and conditions: (i) HO(CH₂)₂NHBoc, TEA, EtOH, 80° C., 15 min, 87%; (ii) 5% Pt/C, MgSO₄, Hz, EtOAc, CH₃OH, rt, 15 h, 86%; (iii) valeryl chloride, TEA, CH₃CN, then TEA, EtOH, 75° C., 24 h, 100%; (iv) CH₃CO2H, EtOH, 25-60° C., 2.5-32 h, 64-94%; (v) 30% NH₄OH/F120, p-TsCl, CH₂C₁₂, 1 h, rt, %; (vi) 4 N HCl, 1,4-dioxane, rt, 1 h, 93%.

### Example 2

### In vitro Activity

UM-2014 was formulated in 10% EtOH/10% DMSO/0.05% Tween 80 (Aq 2) and DOPC/Cholesterol liposomes. These formulations, UM-1007, a phospholipidated oxoadenine TLR7/8 ligand shown below and prepared following Tetrahedron Lett, 2016, 57, 2063-2066, incorporated herein by reference in its entirety, and the corresponding core oxoadenines UM-3007 (for UM-2014) and UM-3002 (for UM-1007), formulated in 2% glycerol/water, were tested in HEK293-hTLR7 and hTLR8 cells and PBMCs.

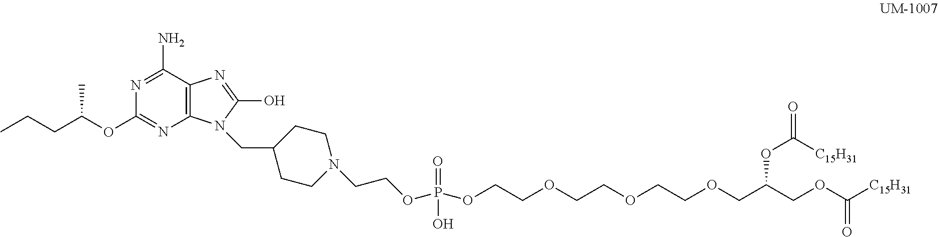

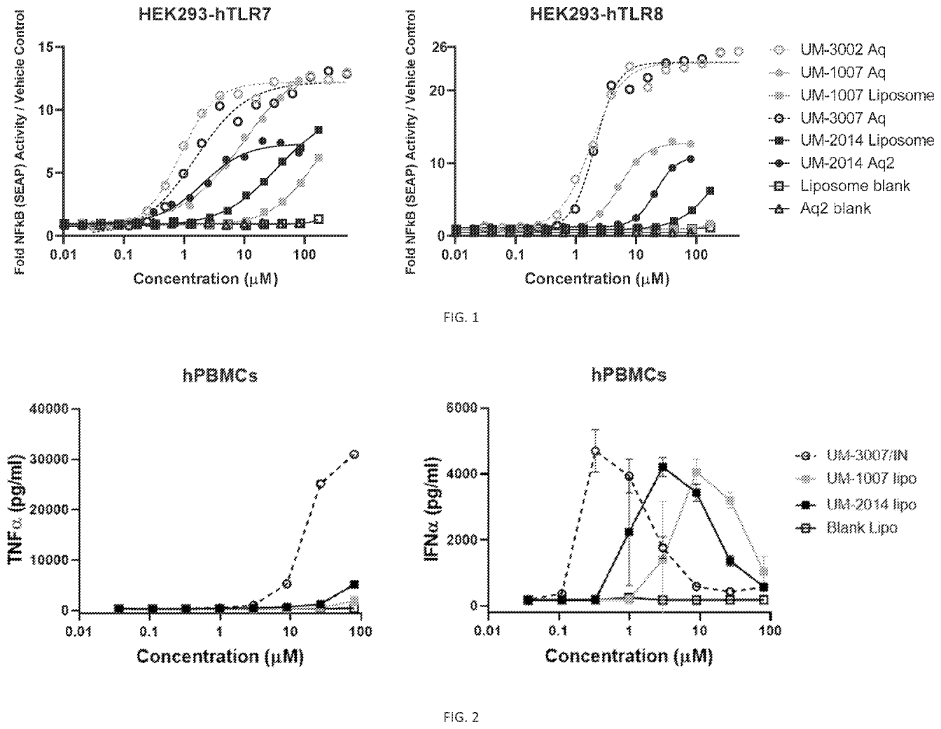

HEK293-hTLR7 and hTLR8 cells. The aqueous formulation of UM-2014 increased NE-kB secreted embryonic alkaline phosphatase (SEAT) production in both HEK293-hTLR7 and hTLR8 cells; the liposomal formulation was less potent than the aqueous formulation (FIG. 1).

Human PBMCs. Liposomal UM-2014 also induced TNFa and IFNa in PBMCs (FIG. 2).

### Example 3

### In vivo Activity

The adjuvanticity of liposomal formulations of UM-2014 (1 and 10 μg doses) was evaluated in an A/Vic murine vaccine model. Mice were intramuscularly immunized at day 0, 14, and 28, with blood collection at day 14 and day 28 (14dp1 and 14dp2, respectively). A tertiary immunization at day 28 was administered to assess cytokine production of antigen-specific T cells in the draining lymph nodes (LN) and spleen 5 days later (day 33).

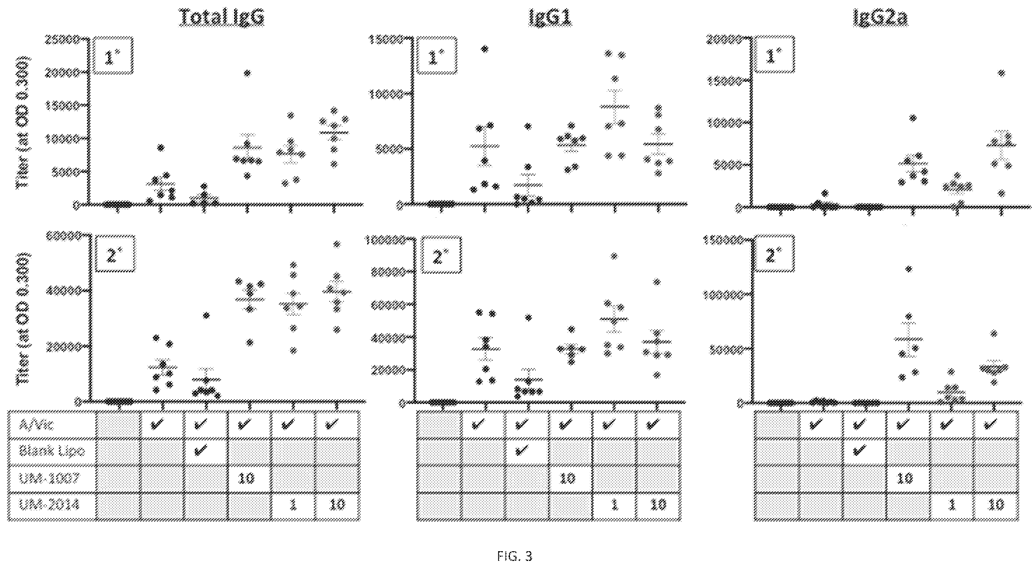

After the primary and secondary immunization, UM-2014 was potent at inducing antibodies (FIG. 3).

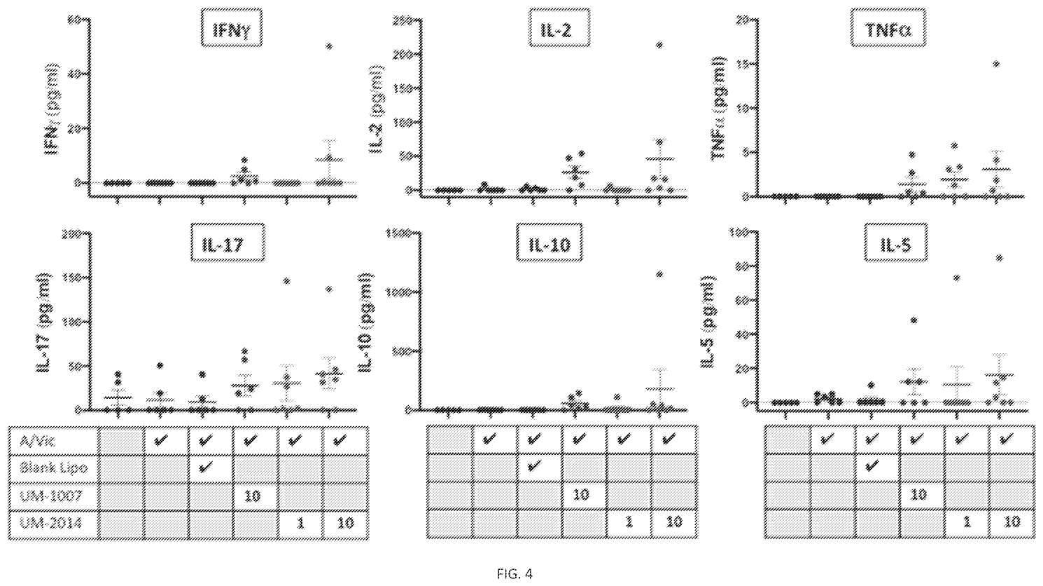

The cytokine secretion by antigen-stimulated T cells from draining LNs and spleen were very similar for mice immunized with UM-2014 and UM-1007 (FIG. 4).

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A compound of Formula (I), 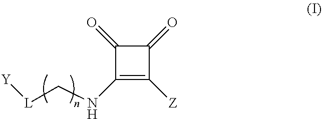
or a pharmaceutically acceptable salt thereof, wherein,
n is an integer from 2 to 6;
Y is 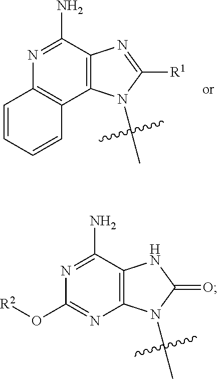
R¹ is hydrogen, C₁-C₆ alkyl, C₁-C₆ alkylamino, or C₁-C₆ alkoxy;
R² is C₁-C₈ alkyl; 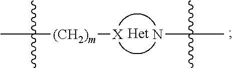
L is a bond or
m is an integer from 1 to 6;
X is CH or N;
Het is a four-, five-, or six-membered saturated heterocycle; 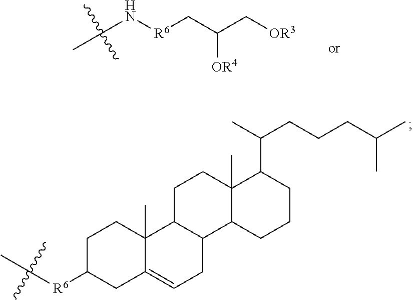
Z is
R³ is C₆-C₂₀ alkyl, C₆-C₂₀ alkenyl, or C(O)R⁵;
R⁴ is hydrogen, C₆-C₂₀ alkyl, C₆-C₂₀ alkenyl, or C(O)R⁵;
R⁵ C₆-C₂₀ alkyl or C₆-C₂₀ alkenyl;
R⁶ is (C₂-C₆ alkylene-O)ₚ or a bond; and
p is 0, 1, 2, 3, 4, 5, 6, 7, 8, 9, 10 ,11, 12, 13, 14, 15, 16, 17, 18, 19 or 20,
wherein 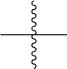
is the point of attachment to formula (I).

2. The compound of claim 1, or a pharmaceutically acceptable salt thereof, wherein R⁶ is (C₂-C₆ alkylene-O)ₚ.

3. The compound of claim 1 or claim 2, or a pharmaceutically acceptable salt thereof, wherein Z is

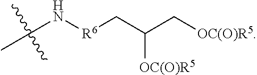

4. The compound of any of claims 1-3, or a pharmaceutically acceptable salt thereof, wherein R⁵ is C₆-C₂₀ alkyl.

5. The compound of any of claims 1-4, or a pharmaceutically acceptable salt thereof, wherein R⁵ is C₁₀-C₂₀ alkyl.

6. The compound of any of claims 1-5, or a pharmaceutically acceptable salt thereof, wherein p is 0, 1, 2, 3, 4, 5, or 6.

7. The compound of any of claims 1-6, wherein Y is 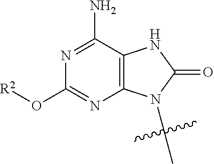
and R² is C₄-C₈ alkyl.

8. The compound of any of claims 1-7, or a pharmaceutically acceptable salt thereof, wherein
L is 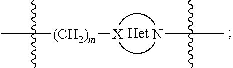
m is 1, 2, 3, 4, or 5;
X is CH or N; and
Het is a five- or six-membered saturated heterocycle.

9. The compound of any of claims 1-8, or a pharmaceutically acceptable salt thereof, wherein L is

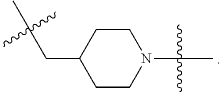 [SMILES: *CC1CCN(*)CC1]

10. The compound of any of claims 1-6, or a pharmaceutically acceptable salt thereof, wherein Y is 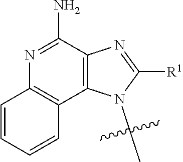
and R¹ is C₁-C₆ alkyl.

11. The compound of any of claims 1-10, or a pharmaceutically acceptable salt thereof, wherein Z is

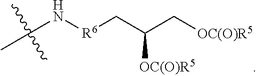

12. The compound of claim 1, or a pharmaceutically acceptable salt thereof, wherein Z is

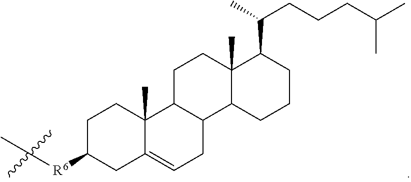

13. The compound of claim 1, wherein the compound is a compound of formula (Ia): 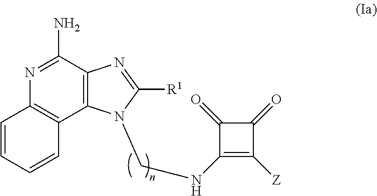
or a pharmaceutically acceptable salt thereof, wherein,
n is an integer from 2 to 6;
R¹ is hydrogen, C₁-C₆ alkyl, C₁-C₆ alkylamino, or C₁-C₆ alkoxy;
Z is 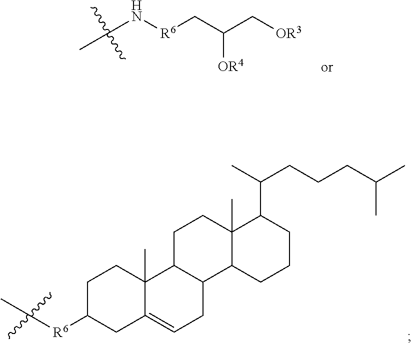
R³ is C₆-C₂₀ alkyl, C₆-C₂₀ alkenyl, or COR⁵;
R⁴ is hydrogen, C₆-C₂₀ alkyl, C₆-C₂₀ alkenyl, or COR⁵;
R⁵ is C₆-C₂₀ alkyl or C₆-C₂₀ alkenyl;
R⁶ is (C₂-C₆ alkylene-O)ₚ or a bond; and
p is 0, 1, 2, 3, 4, 5, 6, 7, 8, 9, 10 ,11, 12, 13, 14, 15, 16, 17, 18, 19 or 20.

14. The compound of claim 13, or a pharmaceutically acceptable salt thereof, wherein R¹ is C₁-C₆ alkyl.

15. The compound of claim 13 or claim 14, or a pharmaceutically acceptable salt thereof, wherein R⁶ is (C₂-C₆ alkylene-O)ₚ.

16. The compound of any of claims 13-15, or a pharmaceutically acceptable salt thereof, wherein Z is

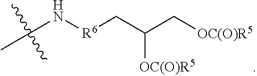

17. The compound of any of claims 13-16 or a pharmaceutically acceptable salt thereof, wherein R⁵ is C₆-C₂₀ alkyl.

18. The compound of any of claims 13-17, or a pharmaceutically acceptable salt thereof, wherein R⁵ is C₁₀-C₂₀ alkyl.

19. The compound of any of claims 13-18, or a pharmaceutically acceptable salt thereof, wherein p is 0, 1, 2, 3, 4, 5, or 6.

20. The compound of any of claims 13-19, or a pharmaceutically acceptable salt thereof, wherein Z is

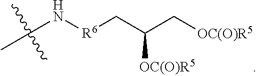

21. The compound of claim 13, or a pharmaceutically acceptable salt thereof, wherein Z is

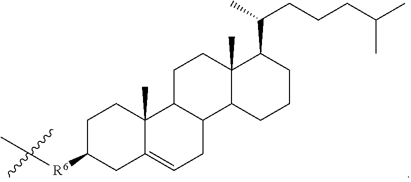

22. The compound of claim 1, wherein the compound is a compound of formula (Ib): 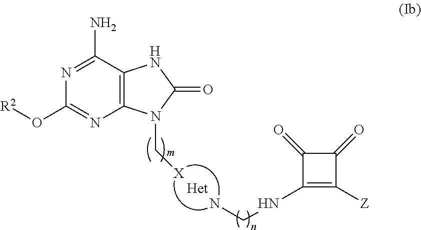
or a pharmaceutically acceptable salt thereof, wherein,
n is an integer from 2 to 6;
R² is C₁-C₈ alkyl;
m is an integer from 1 to 6;
X is CH or N;
Het is a four-, five-, or six-membered saturated heterocycle;
Z is 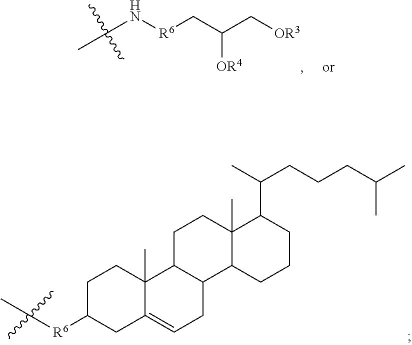
R³ is C₆-C₂₀ alkyl, C₆-C₂₀ alkenyl, or COR⁵;
R⁴ is hydrogen, C₆-C₂₀ alkyl, C₆-C₂₀ alkenyl, or COR⁵;
R⁵ is C₆-C₂₀ alkyl or C₆-C₂₀ alkenyl;
R⁶ is (C₂-C₆ alkylene-O)ₚ or a bond; and
p is 0, 1, 2, 3, 4, 5, 6, 7, 8, 9, 10 ,11, 12, 13, 14, 15, 16, 17, 18, 19 or 20.

23. The compound of claim 22, or a pharmaceutically acceptable salt thereof, wherein L is

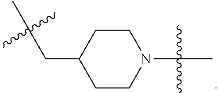 [SMILES: *CC1CCN(*)CC1]

24. The compound of claim 22 or claim 23, or a pharmaceutically acceptable salt thereof, wherein R⁶ is (C₂-C₆ alkylene-O)ₚ.

25. The compound of any of claims 22-24, or a pharmaceutically acceptable salt thereof, wherein Z is

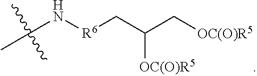

26. The compound of any of claims 22-25 or a pharmaceutically acceptable salt thereof, wherein R⁵ is C₆-C₂₀ alkyl.

27. The compound of any of claims 22-26, or a pharmaceutically acceptable salt thereof, wherein R⁵ is C₁₀-C₂₀ alkyl.

28. The compound of any of claims 22-27, or a pharmaceutically acceptable salt thereof, wherein p is 0, 1, 2, 3, 4, 5, or 6.

29. The compound of any of claims 22-28, or a pharmaceutically acceptable salt thereof, wherein Z is

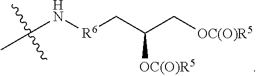

30. The compound of claim 22, or a pharmaceutically acceptable salt thereof, wherein Z is

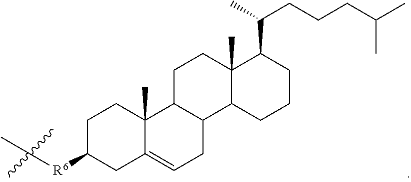

31. A compound selected from the group consisting of: 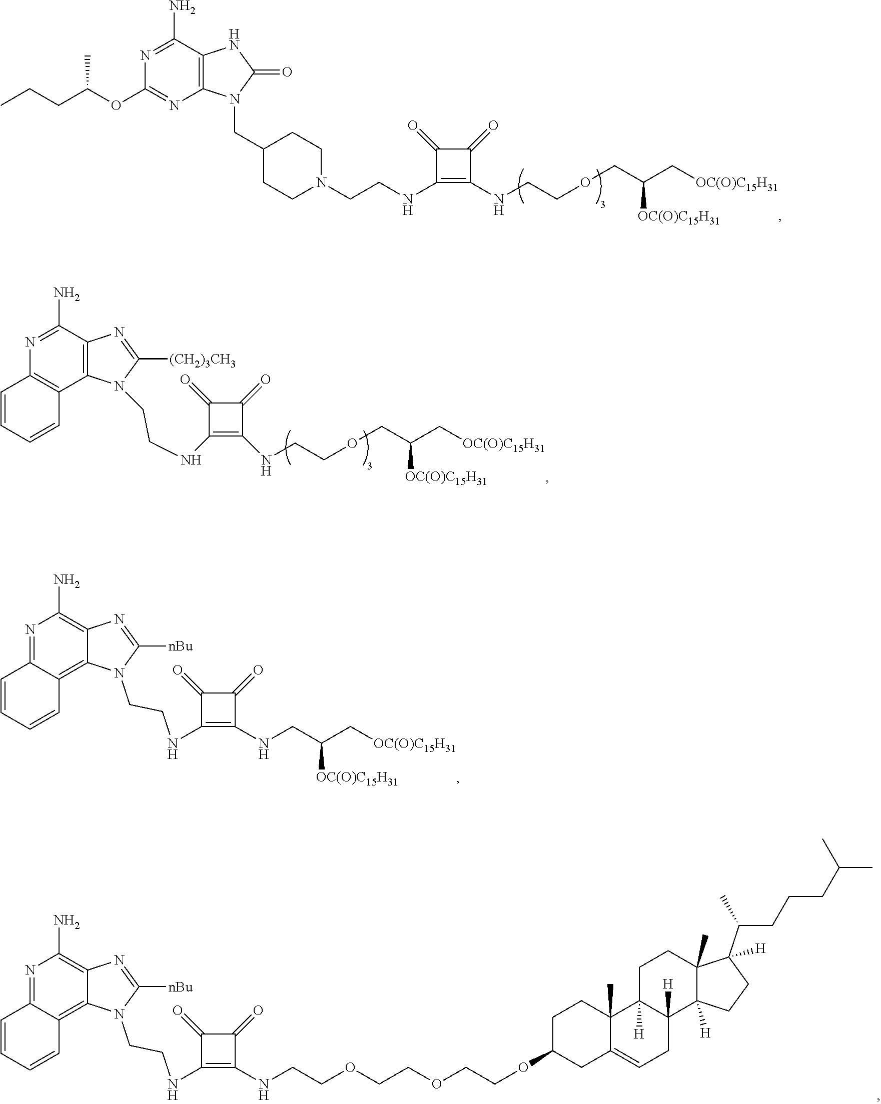
or a pharmaceutically acceptable salt thereof.

32. A pharmaceutical composition comprising the compound of any of claims 1-31, or a pharmaceutically acceptable salt thereof, and a pharmaceutically acceptable carrier.

33. A vaccine composition comprising the compound of any of claims 1-31, or a pharmaceutically acceptable salt thereof, and an antigen.

34. A method for inducing an enhanced immune response in a subject, comprising administering to the subject an effective amount of the compound of any of claims 1-31 or the composition of claim 32 or claim 33.

35. A method of treating a disease or disorder in a subject, comprising administering an effective amount of the compound of any of claims 1-31 or the composition of claim 32 or claim 33.

36. The method of claim 35 wherein the disease or disorder is cancer.
